(19) United States
(12) Patent Application Publication
(10) Pub. No.: US 2023/0228708 A1
(43) Pub. Date: Jul. 20, 2023

(54) ELECTROPHORESIS SYSTEM, ELECTROPHORESIS APPARATUS, AND ELECTROPHORESIS ANALYSIS METHOD

(71) Applicant: SHIMADZU CORPORATION, Kyoto-shi (JP)

(72) Inventors: Kota OGINO, Kyoto-shi (JP); Akira HARADA, Kyoto-shi (JP); Kazunori SHIMIZU, Kyoto-shi (JP); Takashi MORIMOTO, Kyoto-shi (JP)

(73) Assignee: SHIMADZU CORPORATION, Kyoto-shi (JP)

(21) Appl. No.: 18/079,481

(22) Filed: Dec. 12, 2022

(30) Foreign Application Priority Data
Jan. 14, 2022 (JP) 2022-004389

(51) Int. Cl. G01N 27/447

(52) U.S. Cl. CPC G01N 27/44717; G01N 27/44778

(57) ABSTRACT

This electrophoresis system includes an electrophoresis apparatus, an analysis apparatus, and a display unit. Then, in a case where at least one of an apparatus error that is an abnormality in the electrophoresis apparatus or an analysis error that is an abnormality in the analysis of a component of the object-to-be-measured is detected, the analysis apparatus is configured to display, on the display unit, an abnormality detection indication that allows identification of the type of the detected abnormality.

### CROSS-REFERENCE TO RELATED APPLICATIONS

The related application number JP2022-004389, an electrophoresis system, an electrophoresis apparatus, an electrophoresis analysis method, and an electrophoresis analysis program, filed Jan. 14, 2022, by Kota Ogino, Akira Harada, Kazunori Shimizu, and Takashi Morimoto upon which this patent application is based are hereby incorporated by reference.

### BACKGROUND OF THE INVENTION

### Field of the Invention

The present invention relates to an electrophoresis system that performs electrophoresis, an electrophoresis apparatus, and an electrophoresis analysis method.

### Background Art

In the related art, an electrophoresis system that performs electrophoresis is known. Such a system is disclosed, for example, in WO2018/181432.

The electrophoresis system described in WO2018/181432 includes an electrophoresis apparatus and an electrophoresis analysis apparatus. In the electrophoresis apparatus of this electrophoresis system, a DC voltage is applied to electrodes inserted in an electrode reservoir provided at both ends of a capillary that is a channel through which a sample that is an object-to-be-measured is flowed for performing electrophoresis. Then, when the DC voltage is applied to the electrodes and electrophoresis is started, the sample moves by electrophoresis. Then, the capillary is monitored through a detection window, and actual waveform data representing temporal changes in fluorescence brightness from the moving sample is created and output to the electrophoresis analysis apparatus. The electrophoresis analysis apparatus analyzes the actual waveform data output from the electrophoresis apparatus. Specifically, the electrophoresis analysis apparatus detects a peak waveform from the actual waveform data and calculates the amount of DNA.

Here, although not described in WO2018/181432, an abnormality may occur in the operation of the electrophoresis apparatus during the measurement of the object-to-be-measured by electrophoresis. Further, even in a case where there is no abnormality in the operation of the electrophoresis apparatus, an abnormality may occur in the analysis of the component (the amount of DNA) of the object-to-be-measured based on the measurement value measured by the electrophoresis apparatus. In a case where an abnormality in the electrophoresis apparatus or an abnormality in the analysis occurs, an abnormality occurs in the analysis result of the component of the object-to-be-measured. However, even if the analysis result is checked, it is not possible to determine what type of abnormality has occurred. Therefore, in a case where an abnormality occurs in the analysis result of analyzing an object-to-be-measured separated by electrophoresis, it is desired to easily identify the type of abnormality.

### SUMMARY OF THE INVENTION

The present invention has been made to solve the above problems, and one object of the present invention is to provide an electrophoresis system, an electrophoresis apparatus, and an electrophoresis analysis method that can easily identify the type of an abnormality, in a case where the abnormality occurs in an analysis result of analyzing an object-to-be-measured separated by electrophoresis.

In order to achieve the above object, an electrophoresis system according to a first aspect of the present invention includes: an electrophoresis apparatus including a measurement unit that measures an object-to-be-measured separated by electrophoresis in a channel including a separation channel for separating the object-to-be-measured; an analysis apparatus that analyzes a component of the object-to-be-measured separated by electrophoresis, based on a measurement value of the object-to-be-measured that is measured by the measurement unit; and a display unit that displays an analysis result of the object-to-be-measured by the analysis apparatus, in which in a case where at least one of an apparatus error that is an abnormality in the electrophoresis apparatus or an analysis error that is an abnormality in the analysis of the component of the object-to-be-measured is detected, the analysis apparatus is configured to display an abnormality detection indication that allows identification of a type of the detected abnormality, on the display unit.

An electrophoresis apparatus according to a second aspect of the present invention includes: a measurement unit that measures an object-to-be-measured separated by electrophoresis in a channel including a separation channel for separating the object-to-be-measured, in which in a case where at least one of an apparatus error that is an abnormality in the electrophoresis apparatus or an analysis error that is an abnormality in analysis of a component of the object-to-be-measured separated by electrophoresis based on a measurement value of the object-to-be-measured that is measured by the measurement unit is detected, the electrophoresis apparatus is configured to display an abnormality detection indication that allows identification of a type of the detected abnormality, on a display unit.

An electrophoresis analysis method according to a third aspect of the present invention includes: a step of analyzing a component of an object-to-be-measured separated by electrophoresis, based on a measurement value obtained by measuring the object-to-be-measured separated by electrophoresis in a channel including a separation channel for separating the object-to-be-measured; and a step of, in a case where at least one of an apparatus error that is an abnormality in the electrophoresis apparatus that measures the object-to-be-measured separated by electrophoresis or an analysis error that is an abnormality in analysis of the component of the object-to-be-measured is detected, displaying an abnormality detection indication that allows identification of a type of the detected abnormality, on a display unit.

In the electrophoresis system according to the first aspect, the electrophoresis apparatus according to the second aspect, and the electrophoresis analysis method according to the third aspect, in a case where at least one of the apparatus error that is the abnormality in the electrophoresis apparatus or the analysis error that is the abnormality in the analysis of the component of the object-to-be-measured is detected, an abnormality detection indication that allows identification of the type of the detected abnormality is displayed on the display unit. Thus, in a case where an abnormality including at least one of the apparatus error or the analysis error is detected, the type of the detected abnormality can be easily identified by viewing the abnormality detection indication displayed on the display unit. As a result, in a case where an abnormality occurs in the analysis result of analyzing the object-to-be-measured separated by electrophoresis, the type of the abnormality can be easily identified.

### BRIEF DESCRIPTION OF THE DRAWINGS

FIG. 1 is a block diagram showing the overall configuration of an electrophoresis system according to the present embodiment.

FIG. 2 is a schematic diagram for explaining the configuration of an electrophoresis apparatus of the present embodiment.

FIG. 3 is a diagram for explaining the configuration of a chip provided with a channel for electrophoresis.

FIG. 4 is a diagram showing an example of measurement values acquired by measurement of a measurement unit.

FIG. 5 is a diagram showing an example of display on a display unit.

FIG. 6 is a diagram for explaining a well position display.

FIG. 7 is a diagram for explaining a calibration curve.

FIG. 8 is a diagram for explaining gel image display.

FIG. 9 is a diagram for explaining changing an arrangement order of analysis results in the gel image display.

FIG. 10 is a diagram for explaining selection of a plurality of objects-to-be-measured in the well position display.

FIG. 11 is a diagram (flow chart) for explaining an electrophoresis analysis method according to an embodiment.

### DETAILED DESCRIPTION OF THE INVENTION

Hereinafter, an embodiment embodying the present invention will be described with reference to the drawings.

Overall Configuration of Electrophoresis System

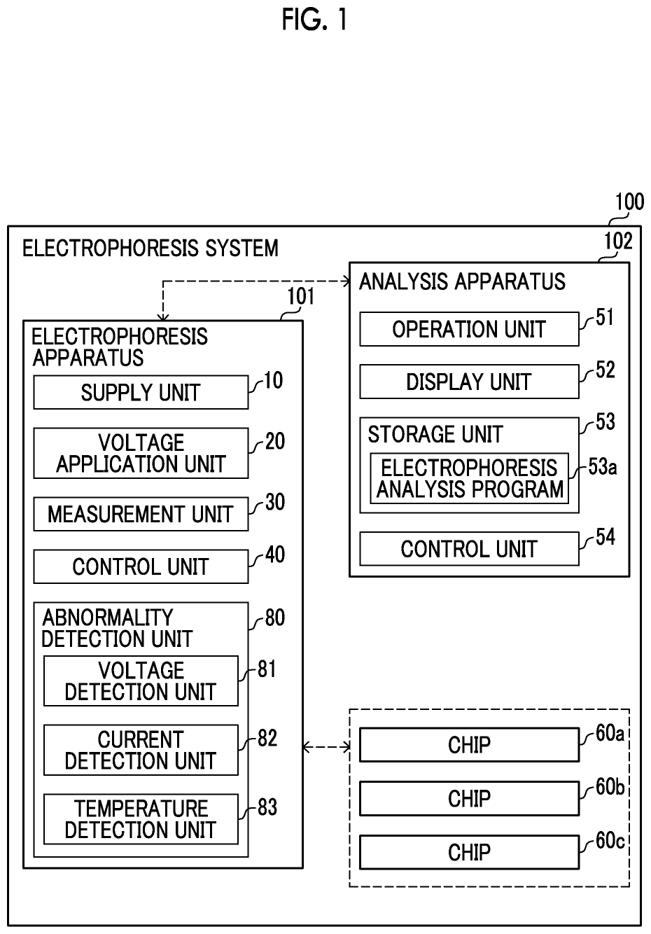

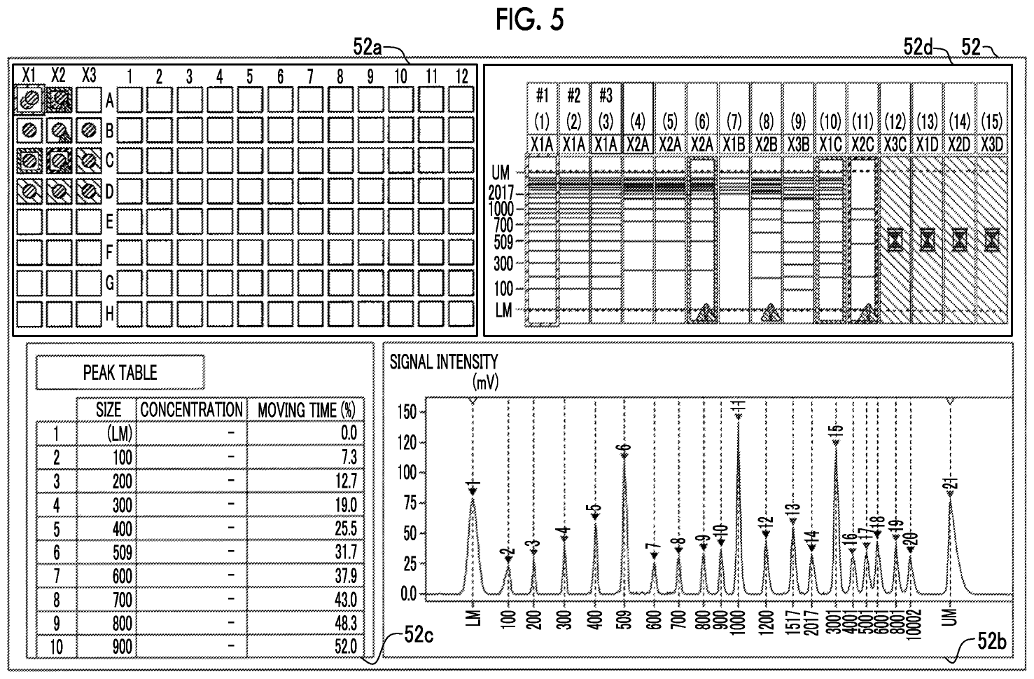

An electrophoresis system 100 according to an embodiment of the present invention is described with reference to FIGS. 1 to 10. In FIGS. 5, 6, and 8 to 10, the difference in color coding is indicated by a difference in hatching.

As shown in FIG. 1, an electrophoresis system 100 according to the present embodiment includes an electrophoresis apparatus 101 and an analysis apparatus 102.

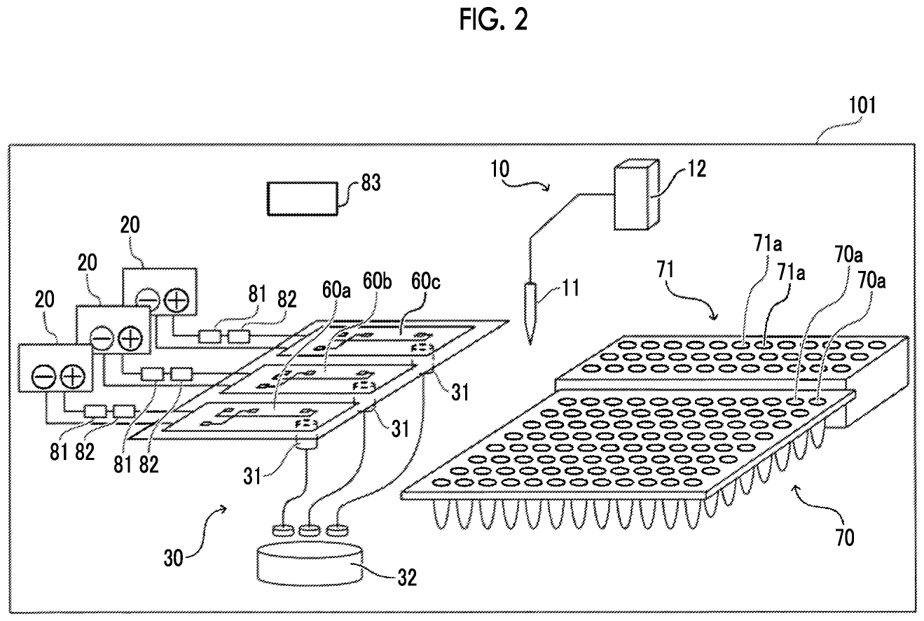

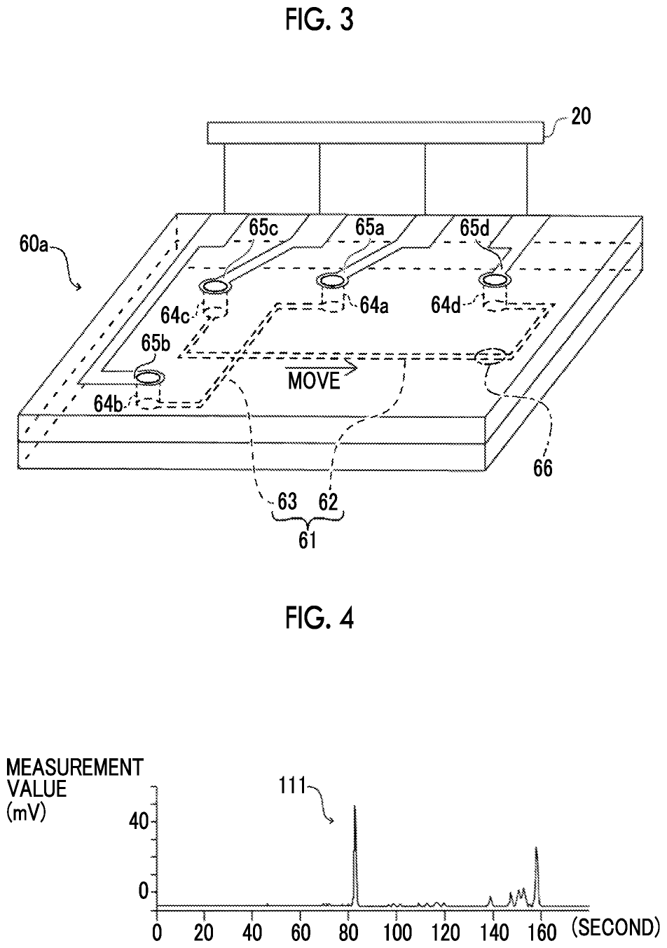

The electrophoresis apparatus 101 separates the object-to-be-measured by electrophoresis by using three chips 60a, 60b, and 60c, thereby measuring components contained in the object-to-be-measured. Specifically, in the electrophoresis apparatus 101, the objects-to-be-measured that are placed in advance on a plate 70 and a sample placement unit 71 (see FIG. 2) are separated by electrophoresis in the channel 61 (see FIG. 3) provided in each of the chips 60a to 60c. Then, the electrophoresis apparatus 101 measures the degree of separation (the degree of distribution of each component) of the object-to-be-measured separated by electrophoresis.

Configuration of Electrophoresis Apparatus

As shown in FIGS. 1 and 2, the electrophoresis apparatus 101 includes a supply unit 10, a voltage application unit 20, a measurement unit 30, and a control unit 40.

In the electrophoresis apparatus 101, the object-to-be-measured and the separation buffer are supplied to the channel 61 of each of the chips 60a, 60b, and 60c by the operation of the supply unit 10 in order to perform measurement by electrophoresis.

Objects-to-be-measured include, for example, deoxyribonucleic acid (DNA), ribonucleic acid (RNA), or protein. The object-to-be-measured includes a sample-to-be-measured (sample) for which the degree of separation of each component by electrophoresis is measured, and a reference sample (size standard) that is a reference for the measurement by electrophoresis of the sample-to-be-measured. The sample-to-be-measured is an object-to-be-measured whose degree of separation by electrophoresis, which is the measurement value 111 (see FIG. 4) measured by the measurement unit 30, is unknown. The reference sample is an object-to-be-measured containing nucleic acids or proteins whose separation characteristics such as molecular weight (chain length) have already been determined. That is, the sample-to-be-measured is an object-to-be-measured with unknown components, and the reference sample is an object-to-be-measured with known components.

Further, the object-to-be-measured is placed on the plate 70 and the sample placement unit 71. The plate 70 is provided with a plurality of wells 70a, which are a plurality of placement positions in which the objects-to-be-measured are placed. For example, the plate 70 is provided with 96 wells 70a arranged in a grid pattern of 8×12. The plate 70 is placed at a plate placement position inside the electrophoresis apparatus 101 by the operator with a plurality of types of objects-to-be-measured placed in all or part of each of the plurality of wells 70a. The object-to-be-measured is placed in the sample placement unit 71 separately from the plate 70. Further, the sample placement unit 71 has wells 71a, which are a plurality of placement positions in which objects-to-be-measured are placed. The wells 71a are arranged in a grid pattern of 3×12.

The separation buffer is a separation medium that fills the channel 61 (see FIG. 3) of each of the chips 60a, 60b, and 60c, before the object-to-be-measured is supplied. The separation buffer includes, for example, at least one of a pH buffer and a water-soluble polymer (cellulosic polymer, or the like). Further, the separation buffer is filled in a buffer container (not shown). Note that the separation buffer may be placed on the plate 70 or the sample placement unit 71. In the electrophoresis apparatus 101, an object-to-be-measured is supplied and electrophoresis is performed in a state in which the channel 61 is filled with a separation buffer in advance.

As shown in FIG. 2, the supply unit 10 has a probe 11 and a pump 12. By moving the probe 11, the supply unit 10 supplies the separation buffer and the object-to-be-measured (sample-to-be-measured and reference sample) placed on the plate 70 or the sample placement unit 71 to the chips 60a to 60c. The pump 12 adjusts the pressure for aspiration and discharge of the separation buffer and the object-to-be-measured in the probe 11.

As shown in FIG. 3, the chips 60a to 60c are each provided with a channel 61 therein. Here, the chips 60a, 60b and 60c have the same configuration. In the following description, the details of the chip 60a will be illustrated and described, and the chips 60b and 60c are the same as the chip 60a, so that the description of thereof will be omitted.

The chip 60a is a microchip for electrophoresis in which a channel 61 for performing electrophoresis is provided inside a pair of combined flat plate-like members. The channel 61 includes a separation channel 62 and a preparation channel 63. The separation channel 62 and the preparation channel 63 are provided so as to cross each other. The separation channel 62 is provided to separate the object-to-be-measured by electrophoresis. Further, the preparation channel 63 is provided to guide the object-to-be-measured to the separation channel 62.

At both ends of the preparation channel 63, reservoir portions 64a and 64b, which are spaces for supply and aspiration of the separation buffer and the object-to-be-measured, are provided. Similarly, both ends of the separation channel 62 are provided with reservoir portions 64c and 64d. Electrodes 65a and 65b are disposed in the reservoir portions 64a and 64b provided at both ends of the preparation channel 63, respectively. Electrodes 65c and 65d are disposed in the reservoir portions 64c and 64d provided at both ends of the separation channel 62, respectively.

In the electrophoresis apparatus 101, electrophoresis is performed by applying a voltage from the voltage application unit 20 to the plurality of electrodes 65a to 65d provided in the channel 61. Further, the magnitude of the voltage applied to the electrodes 65a to 65d is controlled by the control unit 40. Three voltage application units 20 (see FIG. 2) are provided to correspond to the respective chips 60a to 60c so as to apply a DC voltage to the channels 61 of each of the chips 60a to 60c. That is, similar to the chip 60a, with respect to the chips 60b and 60c, a DC voltage is applied to the channel 61 by the voltage application unit 20.

In the electrophoresis apparatus 101, in a case where measurement by electrophoresis is performed in the chip 60a, first, the supply unit 10 fills the entire interior of the channel 61 (the separation channel 62 and the preparation channel 63) with the separation buffer. Then, the supply unit 10 supplies the object-to-be-measured aspirated from, for example, a predetermined well 70a of the plate 70 to the reservoir portion 64a of the preparation channel 63. Then, by applying a predetermined voltage to the electrodes 65a to 65d by the voltage application unit 20, the object-to-be-measured moves inside the preparation channel 63 to a position where the preparation channel 63 and the separation channel 62 intersect. After that, by changing the magnitude of the voltage applied from the voltage application unit 20 to each of the electrodes 65a to 65d, the object-to-be-measured moves toward the electrode 65d (reservoir portion 64d) while being separated by electrophoresis inside the separation channel 62.

At this time, in separation measurement by electrophoresis, the object-to-be-measured moves inside the separation channel 62 at a different speed for each component contained in the object-to-be-measured, depending on the separation characteristics such as the molecular weight (chain length) of the contained component. In the electrophoresis apparatus 101, the separation characteristics of each component of the object-to-be-measured are measured by measuring the components that sequentially reach the measurement position 66 in the separation channel 62. In this manner, in the electrophoresis apparatus 101, the components contained in the object-to-be-measured are measured for each degree of separation (degree of migration).

As shown in FIG. 2, the measurement unit 30 measures an object-to-be-measured separated by electrophoresis in the channel 61 of each of the plurality (three) of chips 60a to 60c. For example, the measurement unit 30 performs fluorescence detection on the component of the object-to-be-measured that has been separated by electrophoresis. The measurement unit 30 has an LED 31 (light emitting diode) that emits excitation light to a measurement position 66 (see FIG. 3) of the separation channel 62. By applying excitation light from the LED 31 to each component of the object-to-be-measured moving in the separation channel 62 while being separated by electrophoresis, each component of the object-to-be-measured is excited and emits fluorescence. The measurement unit 30 measures the components of the object-to-be-measured separated by electrophoresis of the object-to-be-measured, by measuring this fluorescence with the photomultiplier tube 32 via, for example, an optical fiber and a filter member.

As shown in FIG. 4, the photomultiplier tube 32 outputs a measurement signal indicating a measurement value 111 to the control unit 40, according to the detected fluorescence intensity. The measurement value 111 based on the measurement by the measurement unit 30 shows a large value (peak) at the timing when the object-to-be-measured moving while being separated by electrophoresis passes the measurement position 66 (see FIG. 3). Thus, the amount (concentration) and composition (size) are analyzed as the degree of distribution of each component contained in the object-to-be-measured, based on the peak size and position (timing) of each component contained in the object-to-be-measured.

Note that the electrophoresis apparatus 101 is provided with a cleaning mechanism (not shown). The electrophoresis apparatus 101 cleans each part including the chips 60a to 60c and the supply unit 10 each time one object-to-be-measured is measured. The electrophoresis apparatus 101 is configured to repeatedly perform measurements using each of the chips 60a to 60c a plurality of times by cleaning the object-to-be-measured and the separation buffer remaining in the channel 61 with the cleaning mechanism. In this manner, the electrophoresis apparatus 101 sequentially measures each of the plurality of objects-to-be-measured placed in the plurality of wells 70a and 71a.

The control unit 40 controls the operation of each unit of the electrophoresis apparatus 101. The control unit 40 is, for example, a microcomputer (microcontroller) having a processing device such as a central processing unit (CPU) and a storage device such as a flash memory. Further, the control unit 40 includes a communication module and is configured to be able to communicate with the analysis apparatus 102. Then, based on the drive signal from the analysis apparatus 102, the control unit 40 controls the operation of each unit of the electrophoresis apparatus 101 so as to sequentially perform measurement by electrophoresis on a plurality of objects-to-be-measured placed on the plate 70 and the sample placement unit 71.

Specifically, based on the drive signal from the analysis apparatus 102, the control unit 40 operates the supply unit 10 to sequentially supply, for example, the objects-to-be-measured placed in the wells 70a of the plate 70 such that one type is measured each time on each of the chips 60a to 60c. Then, the control unit 40 causes the voltage application units 20 to apply a voltage to the channels 61 of the chips 60a to 60c, thereby separating (moving) the object-to-be-measured by electrophoresis. Further, the control unit 40 acquires the measurement values 111 measured by the measurement unit 30 provided corresponding to each of the chips 60a to 60c. Then, the control unit 40 acquires the measurement value 111, for each of the plurality of wells 70a of the plate 70 and each of the plurality of wells 71a of the sample placement unit 71. Then, the control unit 40 outputs the measurement value 111 of the object-to-be-measured that is measured by the measurement unit 30, for each of the chips 60a to 60c, to the analysis apparatus 102 in real time.

Apparatus Error Detection

Further, as shown in FIGS. 1 and 2, the electrophoresis apparatus 101 includes an abnormality detection unit 80. The abnormality detection unit 80 is configured to detect the abnormality of the electrophoresis apparatus 101. Specifically, the abnormality detection unit 80 includes a voltage detection unit 81, a current detection unit 82, and a temperature detection unit 83. The voltage detection unit 81 detects the voltage output from each of the plurality (three) of voltage application units 20. The current detection unit 82 detects the current flowing through the channel 61 of each of the chips 60a to 60c, based on the voltage applied by the voltage application unit 20. Further, the temperature detection unit 83 detects the internal temperature of the housing in which the chips 60a to 60c and the objects-to-be-measured (the plate 70 and the sample placement unit 71) are placed. The voltage detection unit 81, the current detection unit 82, and the temperature detection unit 83 output detection signals indicating the detected voltage value, current value, and internal temperature to the control unit 40, respectively.

Then, the control unit 40 detects an apparatus error, which is an abnormality of the electrophoresis apparatus 101, based on the detection signal from the abnormality detection unit 80 (the voltage detection unit 81, the current detection unit 82, and the temperature detection unit 83). Further, the control unit 40 is configured to detect two types of apparatus errors, that is, a serious error and a warning error which have different degrees of importance.

Specifically, the control unit 40 stores an abnormality determination threshold value preset in a storage device such as a flash memory and a value of the stable operation range. Then, the control unit 40 detects a serious error, which is an apparatus error having a relatively high degree of importance, in a case where the magnitude of the voltage value, the current value, or the internal temperature detected by the abnormality detection unit 80 is greater than the predetermined abnormality determination threshold value, for example, based on the detection signals sequentially acquired according to the operation of the apparatus. In a case where a serious error is detected, the control unit 40 stops the measurement and stops the operation of the electrophoresis apparatus 101. Further, the control unit 40 detects a warning error which is an apparatus error having a relatively low degree of importance, in a case where the magnitude of the voltage value, the current value, or the internal temperature detected by the abnormality detection unit 80 is not constant (unstable), for example, based on the detection signals sequentially acquired according to the operation of the apparatus. For example, the control unit 40 detects a warning error, in a case where the voltage value, current value, and internal temperature detected during a predetermined period fluctuate beyond a preset predetermined stable operation range. Note that the control unit 40 does not stop the measurement, in a case where a warning error is detected.

The control unit 40 is configured to output an apparatus error signal indicating that an apparatus error has been detected to the analysis apparatus 102, in a case where an apparatus error such as a serious error or a warning error is detected. The apparatus error signal contains information indicating whether a serious error or a warning error has been detected.

Configuration of Analysis Apparatus

As shown in FIG. 1, the analysis apparatus 102 includes an operation unit 51, a display unit 52, a storage unit 53, and a control unit 54. The analysis apparatus 102 is a computer for analyzing the components of the object-to-be-measured separated by electrophoresis, based on the measurement value 111 of the object-to-be-measured that is measured by the electrophoresis apparatus 101. The analysis apparatus 102 is configured to be able to communicate with the electrophoresis apparatus 101, and is configured to acquire the measurement values 111 acquired by the electrophoresis apparatus 101 and the apparatus error signals.

The operation unit 51 receives an input operation by the operator. Further, the operation unit 51 outputs an operation signal based on the received input operation to the control unit 54. The operation unit 51 is, for example, a keyboard and a pointing device such as a mouse.

The display unit 52 is, for example, a monitor such as a liquid crystal display. The display unit 52 displays the information that is input under the control of the control unit 54. In addition, the display unit 52 displays the analysis result of the object-to-be-measured by the control unit 54 of the analysis apparatus 102. The details of the display on the display unit 52 will be described later.

The storage unit 53 is configured by a storage device such as a hard disk drive or a Solid State Drive (SSD). The storage unit 53 stores the measurement values 111 acquired by the electrophoresis apparatus 101. The storage unit 53 also stores an electrophoresis analysis program 53a for operating the control unit 54. The storage unit 53 also stores various parameters such as preset setting values or setting values (measurement conditions) input by the operator.

The control unit 54 is a computer including a CPU, a Random Access Memory (RAM), a Read Only Memory (ROM), and the like. The control unit 54 executes control of each unit of the analysis apparatus 102 by executing a program (electrophoresis analysis program 53a) stored in the storage unit 53. Further, the control unit 54 is configured to be able to communicate with the control unit 40 of the electrophoresis apparatus 101 via a communication module (not shown).

Details of Control by Analysis Apparatus

The control unit 54 transmits drive signals for operating the electrophoresis apparatus 101 to the control unit 40. Specifically, based on an input operation received by the operation unit 51, the control unit 54 acquires various parameters for performing electrophoresis. For example, based on an input operation on the operation unit 51, the control unit 54 acquires well information indicating the wells 70a and 71a in which the objects-to-be-measured (reference sample and sample-to-be-measured) to be measured are placed, measurement condition information including information indicating the magnitude and time of the voltage to be applied, and the like, and schedule information indicating the measurement order of the objects-to-be-measured placed in the plurality of wells 70a and 71a. The well information, the measurement condition information, and the schedule information may be selected from the database stored in the storage unit 53 in advance. Then, the control unit 54 transmits drive signals including the acquired well information, measurement condition information, schedule information, or the like, to the control unit 40 of the electrophoresis apparatus 101. Then, the control unit 54 acquires the measurement value 111 acquired under the control by the control unit 40 based on the transmitted drive signal, from the control unit 40 in real time as the measurement progresses.

Then, as shown in FIG. 5, the control unit 54 analyzes the object-to-be-measured separated by electrophoresis, based on the acquired measurement value 111. Then, the control unit 54 displays the analysis result of the object-to-be-measured, on the display unit 52. Specifically, the control unit 54 analyzes the size (separation index value) of each component of the object-to-be-measured separated by electrophoresis, based on the acquired measurement value 111. For example, in a case where the object-to-be-measured is DNA, the size to be analyzed is represented by the DNA chain length (the number of base pairs). The control unit 54 is configured to display a well position display 52a, a measurement waveform display 52b, a peak table 52c, and a gel image display 52d, on the display unit 52.

The well position display 52a displays the position of each of the plurality of wells 70a and 71a in which each of the plurality of objects-to-be-measured are placed. In the well position display 52a, the positions of the plurality of wells 70a and 71a are shown in a grid pattern so as to correspond to the arrangement of the wells 70a and 71a arranged in a grid pattern. For example, the positions of 96 wells 70a of 8×12 are indicated by squares (rectangles) arranged in a grid pattern of eight vertical columns A to H and 12 horizontal columns of 1 to 12. Further, the positions of the 3×12 wells 71a are indicated by squares (rectangles) arranged in a grid pattern of eight vertical columns A to H and three horizontal columns of X1 to X3.

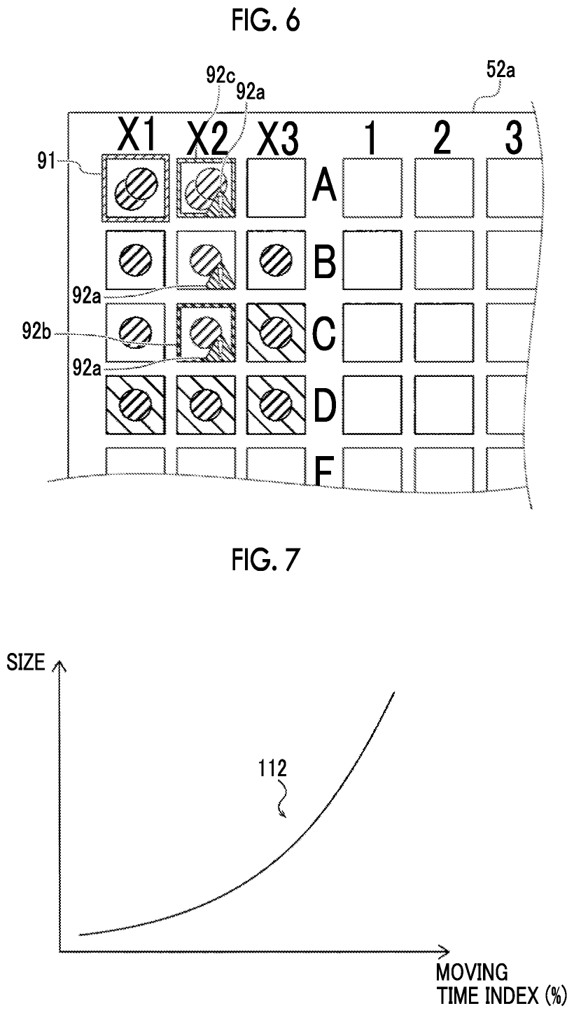

As shown in FIG. 6, in the well position display 52a, the wells 70a or 71a in which the objects-to-be-measured are placed are indicated by displaying a circle inside the squares arranged in a grid pattern. An indication in which two circles are overlapped indicates that the object-to-be-measured placed in the same well 70a or 71a is measured a plurality of times. For example, in the example of well position display 52a in FIG. 6, objects-to-be-measured to be measured a plurality of times are placed at positions indicated by X1A and X2A of the well 71a, and objects-to-be-measured to be measured only once are placed at positions indicated by X1B to X3B, X1C to X3C, and X1D to X3D of the well 71a. Note that how the objects-to-be-measured (reference sample and sample-to-be-measured) are placed in the plurality of wells 70a and 71a is set based on the input operation on the operation unit 51 or the data stored in the storage unit 53. For example, objects-to-be-measured, which are different types of reference samples, are placed at the positions indicated by X1A and X2A of the well 71a. At the positions X1B to X3B, X1C to X3C, and X1D to X3D of the well 71a, objects-to-be-measured, which are samples-to-be-measured of unknown size, are placed.

Further, as shown in FIG. 5, the measurement waveform display 52b is a waveform (electropherogram) showing time-series values of the acquired measurement values 111. Specifically, the measurement waveform display 52b is represented by the size on the horizontal axis and the signal intensity (measurement value 111) measured by the measurement unit 30 on the vertical axis, based on the time-series values of the acquired measurement value 111. The measurement waveform display 52b also displays a numerical value indicating the size of the component of the separated object-to-be-measured.

Here, the object-to-be-measured separated by electrophoresis is mixed with an internal standard marker substance that is a reference in the analysis of the components of the object-to-be-measured. That is, in the measurement by electrophoresis, the channel 61 is supplied with an internal standard marker substance, which serves as a reference for the minimum and maximum sizes (chain lengths) measured by electrophoresis, together with the object-to-be-measured. Specifically, the internal standard marker substance is placed in each of the wells 70a and 71a, in a state of being mixed with each object-to-be-measured. The internal standard marker substance has a Lower Marker (hereinafter referred to as LM) and an Upper Marker (hereinafter referred to as UM). LM is measured as a size of a sufficiently smaller value compared to the object-to-be-measured in the measurement by the measurement unit 30. Then, the UM is measured as a size of a sufficiently larger value compared to the object-to-be-measured in the measurement by the measurement unit 30. That is, the LM is sufficiently smaller in size than the reference sample and the sample-to-be-measured, and the UM is sufficiently larger in size than the reference sample and the sample-to-be-measured.

In the measurement by electrophoresis, common LM and UM are mixed for both the reference sample and the sample-to-be-measured. Then, based on the LM and UM measured in a case where the reference sample is measured and the LM and UM measured in a case where the sample-to-be-measured is measured, the size of the sample-to-be-measured is analyzed, by comparing the timings at which the measurement value 111 of the reference sample and the measurement value 111 of the sample-to-be-measured are measured. It is set in advance which of the plurality of objects-to-be-measured placed in the wells 70a and 71a is the reference sample. Further, in a case where a plurality of reference samples are placed in the well 70a or 71a, it is preset for each object-to-be-measured which reference sample is used for the analysis of the sample-to-be-measured.

Specifically, as shown in FIG. 7, in a case of analyzing the measurement by electrophoresis of the object-to-be-measured, a calibration curve 112 is created based on the measurement values 111 acquired by measuring the reference sample. Specifically, first, measurement by electrophoresis of a reference sample, which is a reference for the object-to-be-measured, is performed. Then, the analysis apparatus 102 detects a peak from the waveform of the measurement value 111 of the measured reference sample. Then, based on the detected LM, UM, and each peak of each component, with the size of LM is 0 and the size of UM is a predetermined value, a ratio between the time (timing) when the lower marker (LM) and the upper marker (UM) detected by the measurement unit 30 and the time (timing) when each component contained in the reference sample whose size is known in advance is detected by the measurement unit 30 is acquired, and a calibration curve 112 is generated with the horizontal axis as “moving time index” and the vertical axis as “size”. For example, in the measurement of the object-to-be-measured by the measurement unit 30, with the time (timing) at which the LM is measured is 0 and the time (timing) at which the UM is measured is 100, the moving time index is an index that expresses the time (timing) at which each component of the object-to-be-measured is measured, as a percentage.

Then, as shown in FIG. 5, the analysis apparatus 102 (the control unit 54) analyzes the size of the sample-to-be-measured, based on the measurement value 111 acquired by electrophoresis in a state where a sample-to-be-measured, which is an object-to-be-measured of unknown size, is mixed with LM and UM and the generated calibration curve 112. Specifically, the analysis apparatus 102 detects peaks, from the waveform generated based on the measurement value 111 acquired by measuring a sample-to-be-measured of unknown size. Then, the analysis apparatus 102 detects LM and UM from the detected peaks, and analyzes the size corresponding to the peak from the calibration curve 112 of the reference sample, based on the relative time ratio (moving time index) of the peaks included between LM and UM. Then, the analysis apparatus 102 displays the size corresponding to each detected peak, in the measurement waveform display 52b. Further, the analysis apparatus 102 displays the specific numerical values of the sizes and moving time indices corresponding to the detected peaks in the peak table 52c.

The gel image display 52d shows an analysis result (size) of each of the plurality of objects-to-be-measured. Specifically, in the gel image display 52d, for each measurement of the object-to-be-measured using the chips 60a to 60c, an indication (image) indicating the distribution of the component (size) of each of the plurality of objects-to-be-measured, analyzed by the analysis apparatus 102, is displayed side by side as a plurality of analysis results. In the analysis result in the gel image display 52d, a plurality of horizontal bars (band pattern, ladder) represent the size of each analyzed component of the object-to-be-measured. Further, in the analysis result in the gel image display 52d, a plurality of horizontal bars are arranged at positions corresponding to the peaks of the waveform of the measurement value 111, according to size, with LM as the lower end and UM as the upper end. Further, in the gel image display 52d, the position indicating the LM and the position indicating the UM are shown to be common positions in the plurality of analysis results displayed side by side. For example, in the analysis result in the gel image display 52d, the magnitude of the measurement value 111 is represented by the shade of color, by setting the pixel value according to the magnitude of the measurement value 111 (signal intensity).

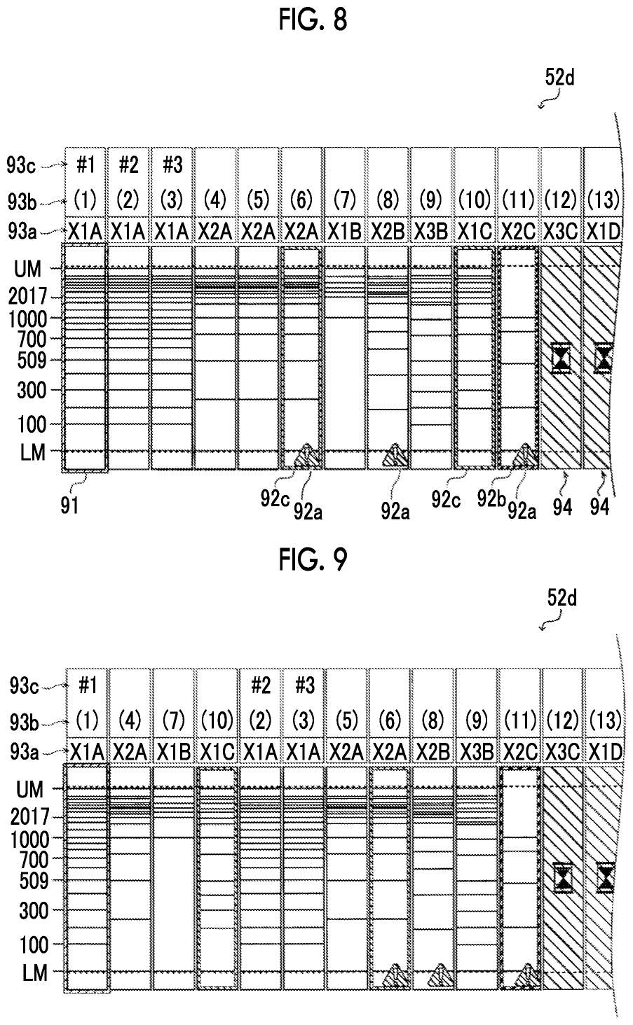

Further, as shown in FIG. 8, in the present embodiment, the gel image display 52d displays a well number 93a indicating the well 70a or 71a in which the object-to-be-measured corresponding to each analysis result is placed, and a measurement order number 93b indicating the measurement order. A well number 93a and a measurement order number 93b are displayed for each measurement by electrophoresis (each measurement result). The well number 93a is represented by the notation common to the positions of the wells 70a and 71a in the well position display 52a. The measurement order number 93b indicates the order of measurement by the electrophoresis apparatus 101. The analysis apparatus 102 arranges and displays a plurality of analysis results horizontally in ascending order of the measurement order number 93b, for example, in the gel image display 52d.

Further, the analysis apparatus 102 acquires the measurement value 111 from the electrophoresis apparatus 101 in real time according to the progress of the measurement, and analyzes the object-to-be-measured for which the measurement has been completed, each time the measurement of one object-to-be-measured is completed. Then, the analysis apparatus 102 displays images showing the analysis results in the gel image display 52d in order from the object-to-be-measured for which the measurement has been completed. In addition, in a case where the object-to-be-measured placed in the same well 70a or 71a is measured a plurality of times, the analysis results created for each measurement are displayed side by side.

Further, the analysis apparatus 102 is configured to display a chip number 93c in the gel image display 52d. The chip number 93c is a number indication of any of 1 to 3 indicating each of the chips 60a to 60c. Then, in the gel image display 52d, in the analysis of the measurement values 111 acquired by the measurements of each of the chips 60a to 60c, only the analysis result of the reference sample that is the reference for generating the calibration curve 112 is indicated by the chip number 93c.

Note that, as shown in FIG. 5, the analysis apparatus 102 (control unit 54) is configured to display, on the display unit 52, a measurement waveform display 52b and a peak table 52c corresponding to one analysis result selected from among a plurality of analysis results in the gel image display 52d. Specifically, the operation unit 51 receives a selection operation of selecting one analysis result from images showing a plurality of analysis results displayed side by side in the gel image display 52d. The analysis apparatus 102 is configured to display, on the display unit 52, a measurement waveform display 52b and a peak table 52c corresponding to one selected analysis result, based on a selection operation received by the operation unit 51. Note that character information indicating the positions of the wells 70a and 71a corresponding to the selected analysis result, the type of object-to-be-measured (reference sample or sample-to-be-measured), and the number of the chips 60a to 60c used for measurement may be displayed on the display unit 52.

Further, as shown in FIGS. 6 and 8, the analysis apparatus 102 displays a selection indication 91 indicating one selected analysis result on the display unit 52. In the gel image display 52d, the selection indication 91, which is a blue frame surrounding the outside of the selected one analysis result, is displayed. In the well position display 52a, the selection indication 91 is displayed to indicate a square of the position corresponding to the well 70a or 71a in which the object-to-be-measured corresponding to the selected one analysis result is placed. The selection indication 91 in the well position display 52a is displayed as a blue frame surrounding the outside of the displayed square, similarly to the gel image display 52d.

Standby Indication

Further, as shown in FIG. 8, in the present embodiment, the analysis apparatus 102 is configured to display, on the display unit 52, a standby indication 94 indicating an object-to-be-measured waiting to be measured among a plurality of objects-to-be-measured. Specifically, the analysis apparatus 102 displays, in the gel image display 52d, a standby indication 94 so as to indicate an object-to-be-measured that has not yet been measured and is scheduled to be measured. The standby indication 94 is displayed side by side in the order of measurement in the same manner as the image showing the analysis result of the object-to-be-measured for which the analysis has been completed. The standby indication 94 includes, for example, an hourglass icon image. Further, in the well position display 52a, the analysis apparatus 102 colors the square at the position corresponding to the well 70a or 71a in which the object-to-be-measured for which the measurement has been completed is placed, with a gray background color, and colors the square at the position corresponding to the well 70a or 71a in which the object-to-be-measured waiting to be measured is placed, with a blue background color. Further, in the gel image display 52d, the analysis apparatus 102 colors the standby indication 94 indicating an object-to-be-measured waiting for measurement, with a blue background color, similar to the well position display 52a. In addition, in the well position display 52a, the analysis apparatus 102 blinks the circular display inside the square at the corresponding position so as to the well 70a or 71a where the object-to-be-measured is placed and is currently being measured using the chips 60a to 60c.

Abnormality Detection Indication

Further, the control unit 54 is configured to detect an analysis error, in a case where an abnormality occurs in the analysis of the object-to-be-measured. For example, in the analysis of the reference sample, the control unit 54 detects an analysis error, in a case where it is not possible to generate the calibration curve 112 because a peak cannot be detected from the acquired measurement value 111 (measurement waveform display 52b), or LM or UM cannot be detected. Similarly, in a case where the calibration curve 112 of the set reference sample is not generated in the analysis of the sample-to-be-measured, or in a case where the size cannot be calculated based on the acquired measurement value 111, the control unit 54 detects an analysis error. In other words, the analysis error is also acquired in a case where the electrophoresis apparatus 101 operates normally, separately from the apparatus error caused by the electrophoresis apparatus 101 described above.

Then, as shown in FIGS. 6 and 8, in the present embodiment, the analysis apparatus 102 (control unit 54) is configured to display an abnormality detection indication that allows identification of the type of the detected abnormality on the display unit 52, when at least one of an apparatus error or an analysis error is detected in any of the plurality of objects-to-be-measured. The abnormality detection indication includes at least one of an analysis error indication 92a, a serious error indication 92b, and a warning error indication 92c. Specifically, in at least one of a case where an apparatus error signal is acquired from the electrophoresis apparatus 101 or a case where an analysis error is detected in the analysis of the object-to-be-measured, the analysis apparatus 102 is configured to display, in the well position display 52a and the gel image display 52d displayed on the display unit 52, the abnormality detection indication such that the object-to-be-measured in which an abnormality is detected can be identified. The abnormality detection indication is displayed for each of the plurality of wells 70a and 71a arranged in a grid pattern in the well position display 52a. Further, the abnormality detection indication is displayed for each of the plurality of analysis results displayed side by side, in the gel image display 52d.

Further, in the present embodiment, the abnormality detection indication is displayed in a different display mode depending on the type of abnormality detected. That is, in the abnormality detection indication, the mode of display differs depending on whether an analysis error is detected or an apparatus error is detected. Further, in the abnormality detection indication, the mode of display differs depending on the degree of importance of the detected apparatus error (a serious error and a warning error). The abnormality detection indication is displayed such that the type of abnormality can be identified by color-coding according to the type of abnormality detected and an icon image indication.

Specifically, the analysis error indication 92a is an icon image indication indicating that an analysis error has been detected in the analysis of the object-to-be-measured. The analysis error indication 92a is an icon image displayed in the lower right portion of a square (rectangle) indicating the position of the well 70a or 71a in which the object-to-be-measured in which the analysis error has been detected is placed, in the well position display 52a. The analysis error indication 92a has a substantially triangular shape, and an exclamation mark (“!”) is displayed inside. Further, the substantially triangular background portion has a yellow background color. Similarly, in the gel image display 52d, the analysis error indication 92a is displayed in the lower right portion of the analysis result of the object-to-be-measured in which the analysis error is detected, among the plurality of analysis results displayed side by side.

The serious error indication 92b is an indication indicating that an apparatus error signal indicating that a serious error among the apparatus errors has been detected has been acquired from the electrophoresis apparatus 101, in the measurement of the object-to-be-measured. Then, the warning error indication 92c is an indication indicating that an apparatus error signal indicating that a warning error among the apparatus errors has been detected has been acquired from the electrophoresis apparatus 101, in the measurement of the object-to-be-measured. A serious error indication 92b and a warning error indication 92c are displayed as a color-coded frame according to degree of importance of the apparatus error in the well position display 52a inside a square (rectangle) indicating the position of the well 70a or 71a in which the object-to-be-measured in which the apparatus error has been detected is placed. For example, in a case where a serious error is detected, a serious error indication 92b, which is a red frame, is displayed. Then, in a case where a warning error is detected, a warning error indication 92c, which is a yellow frame, is displayed. Similarly, in the gel image display 52d, the serious error indication 92b, which is a red frame, is displayed in the inner part of the analysis result of the object-to-be-measured in which a serious error is detected, and the warning error indication 92c, which is a yellow frame, is displayed in the inner part of the analysis result of the object-to-be-measured in which a warning error is detected.

In addition, in a case where both a serious error and a warning error among apparatus errors are detected in the measurement of the same object-to-be-measured, only the serious error indication 92b is displayed and the warning error indication 92c is not displayed, in both the well position display 52a and the gel image display 52d. Further, in a case where both an analysis error and an apparatus error are detected in the measurement and analysis of the same object-to-be-measured, both the analysis error indication 92a and the serious error indication 92b or warning error indication 92c are displayed at the same time.

Further, in the gel image display 52d, in a case where an analysis result in which an abnormality detection indication including at least one of the analysis error indication 92a and the serious error indication 92b or the warning error indication 92c is displayed is selected, the abnormality detection indication and the selection indication 91 are simultaneously displayed on the selected analysis result. Similarly, in the well position display 52a, the abnormality detection indication and the selection indication 91 are simultaneously displayed in the indication indicating the position of the well 70a or

Rearrangement of Gel Image Display

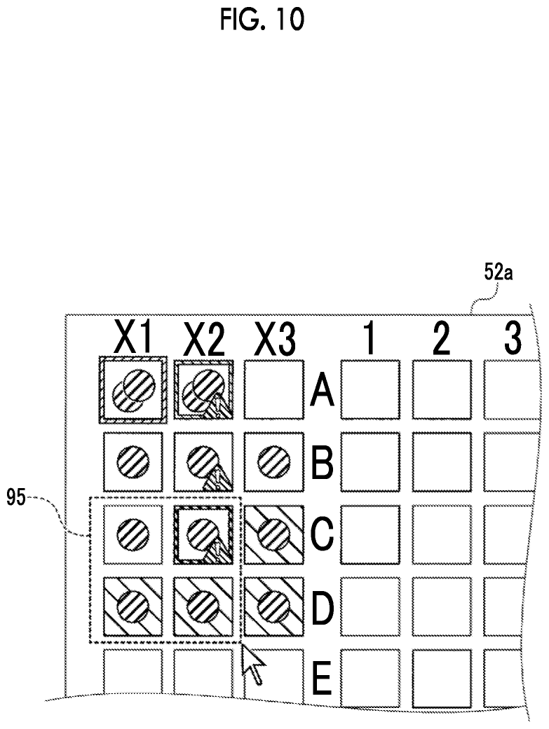

As shown in FIGS. 8 and 9, in the present embodiment, the control unit 54 is configured to be able to change the arrangement order of the plurality of analysis results displayed side by side in the gel image display 52d, based on the operation received by the operation unit 51. For example, the arrangement order in the gel image displays 52d is changed by a drag operation using a pointing device such as a mouse of the operation unit 51. At this time, along with the movement due to the change in the arrangement order in the gel image display 52d, the abnormality detection indication, and the indications of the well number 93a, the measurement order number 93b, and the chip number 93c also move in the same manner as the corresponding analysis results.

Selection of Plurality of Wells

Further, in the analysis apparatus 102, the control unit 54 is configured to be able to selectively output analysis results of a plurality of objects-to-be-measured placed in the wells 70a and 71a. For example, based on the input operation received by the operation unit 51, the control unit 54 outputs the analysis result of the selected object-to-be-measured, from among the objects-to-be-measured placed in the plurality of wells 70a and 71a to a storage device (not shown) provided separately from the analysis apparatus 102.

As shown in FIG. 10, the analysis apparatus 102 is configured to select the analysis result of each of the plurality of objects-to-be-measured placed in the plurality of wells 70a and 71a included in the predetermined area 95, based on the reception of a selection operation by the operation unit 51 to surround a predetermined area 95 from among the plurality of wells 70a and 71a arranged in a grid pattern on the well position display 52a. For example, the predetermined area 95 is selected by a range selection operation using a pointing device such as a mouse of the operation unit 51.

Regarding Electrophoresis Analysis Method

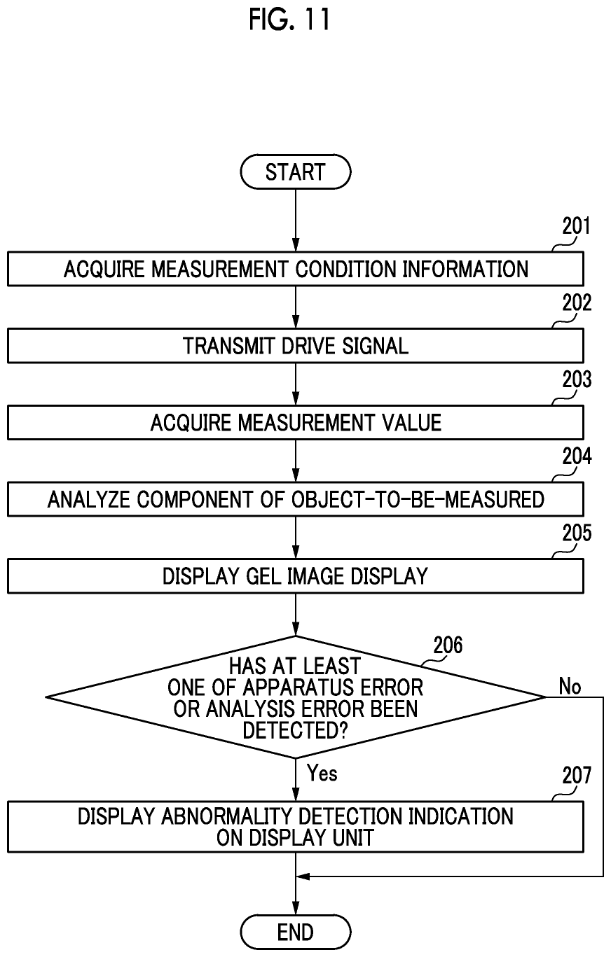

Next, an electrophoresis analysis method using the electrophoresis system 100 according to the present embodiment will be described with reference to FIG. 11. The control process in steps 201 to 207 is performed by executing the electrophoresis analysis program 53a stored in the storage unit 53 by the control unit 54 (analysis apparatus 102).

First, in step 201, measurement condition information for performing measurement is acquired. Specifically, well information indicating the wells 70a and 71a in which the objects-to-be-measured to be measured are placed, schedule information indicating the order of measurement, information indicating types of objects-to-be-measured (reference sample and object-to-be-measured) placed in the wells 70a and 71a, or the like are acquired (set) along with measurement condition information including information indicating the magnitude and time of the voltage applied by the voltage application unit 20. These pieces of information may be acquired based on an input operation on the operation unit 51 or may be acquired from information stored in advance in the storage unit 53 or the like.

Next, in step 202, a drive signal including the acquired measurement condition information is transmitted to the control unit 40 of the electrophoresis apparatus 101. The drive signal includes the acquired well information, schedule information, and the like, in addition to the measurement condition information. Based on this drive signal, in the electrophoresis apparatus 101, measurement by electrophoresis using the chips 60a to 60c is performed for each object-to-be-measured in the predetermined wells 70a and 71a in the set order.

Next, at step 203, the measurement value 111 is acquired based on the measurement by the measurement unit 30 of the electrophoresis apparatus 101. The measurement values 111 are acquired sequentially in real time as the measurement progresses.

Next, in step 204, an analysis based on the acquired measurement values 111 is performed. Specifically, based on the acquired measurement values 111, the component of the object-to-be-measured separated by electrophoresis is analyzed.

Next, in step 205, the gel image display 52d is displayed on the display unit 52 based on the analysis of the component of the object-to-be-measured. Further, the gel image display 52d is displayed on the display unit 52 together with the well position display 52a, the measurement waveform display 52b, and the peak table 52c.

Next, in step 206, it is determined whether or not at least one of an apparatus error or analysis error has been detected. In a case where it is determined that at least one of the apparatus error or the analysis error is detected, the process proceeds to step 207. In a case where it is not determined that at least one of the apparatus error or the analysis error is detected, the control process is terminated.

In step 207, when at least one of the apparatus error or the analysis error is detected, an abnormality detection indication that allows identification of a type of the detected abnormality is displayed on the display unit 52. Specifically, in a case where an analysis error is detected, an analysis error indication 92a, which is an icon image indicating an analysis error, is displayed on the corresponding analysis result in the gel image display 52d and the position indicating the well 70a or 71a in which the corresponding object-to-be-measured is placed in the well position display 52a. Then, in a case where an apparatus error is detected, depending on the degree of importance of the apparatus error, the serious error indication 92b or the warning error indication 92c is displayed on the corresponding analysis result in the gel image display 52d and the position indicating the well 70a or 71a in which the corresponding object-to-be-measured is placed in the well position display 52a.

Note that the control process in steps 203 to 207 is executed each time an object-to-be-measured disposed in the well 70a or 71a is measured in one chip 60a (60b or 60c). That is, in a case where the measurement and analysis for the object-to-be-measured placed in the predetermined well 70a or 71a are completed, the measurement and analysis for a new object-to-be-measured placed in the next well 70a or 71a are performed. Further, the respective measurements in the chips 60a-60c are performed simultaneously.

### Effect of Present Embodiment

In the present embodiment, the following effects can be obtained.

In the electrophoresis system 100 and the electrophoresis apparatus 101 of the present embodiment, as described above, in a case where at least one of an apparatus error that is an abnormality in the electrophoresis apparatus 101 and an analysis error that is an abnormality in the analysis of the component of the object-to-be-measured is detected, an abnormality detection indication that allows identification of the type of the detected abnormality is displayed on the display unit 52. Thus, in a case where an abnormality including at least one of an apparatus error and an analysis error is detected, the type of the detected abnormality can be easily identified by viewing the abnormality detection indication displayed on the display unit 52. As a result, in a case where an abnormality occurs in the analysis result of analyzing the object-to-be-measured separated by electrophoresis, the type of the abnormality can be easily identified.

Further, in the above-described embodiment, further effects can be obtained by configuring as follows.

That is, in the present embodiment, as described above, the electrophoresis apparatus 101 is configured to output an apparatus error signal indicating that an apparatus error has been detected to the analysis apparatus 102, and the analysis apparatus 102 is configured to display an abnormality detection indication on the display unit 52, in at least one of a case where an apparatus error signal is acquired from the electrophoresis apparatus 101 and a case where an analysis error is detected in the analysis of the component of the object-to-be-measured. With this configuration, the operator who performs the measurement operation can easily recognize at least one of detection of an apparatus error by the electrophoresis apparatus 101 and detection of an analysis error in the analysis by the analysis apparatus 102, by viewing the abnormality detection indication displayed on the display unit 52 by the analysis apparatus 102. As a result, by viewing the abnormality detection indication displayed on the display unit 52, the operator can easily recognize whether or not an apparatus error and an analysis error have been detected.

Further, in the present embodiment, as described above, the analysis apparatus 102 is configured to display an abnormality detection indication with a different display mode depending on the type of detected abnormality on the display unit 52, in a case where at least one of an apparatus error and an analysis error is detected. With this configuration, since the abnormality detection indication is displayed in a different display mode depending on the type of detected abnormality, the operator can easily and intuitively identify the type of detected abnormality, by viewing the difference in the display mode of the abnormality detection indication.

Further, in the present embodiment, as described above, the analysis apparatus 102 is configured to display an abnormality detection indication with a different display mode depending on the degree of importance of the detected apparatus error on the display unit 52, in a case where an apparatus error is detected. With this configuration, since the abnormality detection indication is displayed in a different display mode depending on the degree of importance of the apparatus error, the operator can easily identify the type of detected apparatus error and intuitively and easily identify the degree of importance of the apparatus error, by viewing the abnormality detection indication, in a case where an abnormality occurs in the electrophoresis apparatus 101.

Further, in the present embodiment, as described above, the analysis apparatus 102 is configured to display, on the display unit 52, an abnormality detection indication that allows identification of the type of abnormality by color-coding according to the type of detected abnormality and icon image indication, in a case where at least one of an apparatus error and an analysis error is detected. With this configuration, in a case where at least one of an apparatus error and an analysis error is detected, the abnormality detection indication that allows identification of the type of abnormality by at least one of color coding according to the type of detected abnormality and an icon image indication is displayed on the display unit 52, the operator can intuitively and easily identify the type of the detected abnormality, by viewing at least one of the color coding or the icon image indication in the abnormality detection indication.

Further, in the present embodiment, as described above, the electrophoresis apparatus 101 is configured to measure a plurality of objects-to-be-measured, and the analysis apparatus 102 is configured to display an abnormality detection indication such that the object-to-be-measured in which the abnormality is detected can be identified, in a case where at least one of an apparatus error or an analysis error in any of the plurality of objects-to-be-measured is detected. With this configuration, in a case where an abnormality is detected in any one of the plurality of objects-to-be-measured, the operator can easily recognize in which object-to-be-measured an abnormality has been detected, and easily recognize the type of abnormality that has been detected, by viewing the abnormality detection indication displayed on the display unit 52.

Further, in the present embodiment, as described above, the electrophoresis apparatus 101 is configured to sequentially measure a plurality of objects-to-be-measured, and the analysis apparatus 102 is configured to display, on the display unit 52, a standby indication 94 showing objects-to-be-measured waiting for measurement, among the plurality of objects-to-be-measured. With this configuration, it is possible to easily distinguish between an object-to-be-measured for which measurement has been completed and an object-to-be-measured for which measurement is to be performed from among the plurality of objects-to-be-measured. As a result, it is possible to easily identify an object-to-be-measured in which an abnormality has been detected from among a plurality of objects-to-be-measured while distinguishing objects-to-be-measured for which measurement has not yet been performed.

Further, in the present embodiment, as described above, the analysis apparatus 102 is configured to display, on the display unit 52, the well position display 52a indicating the position of each of the plurality of wells 70a and 71a in which each of the plurality of objects-to-be-measured is disposed and a gel image display 52d showing the analysis result of each of the plurality of objects-to-be-measured, and display an abnormality detection indication such that the object-to-be-measured in which an abnormality is detected can be identified, in the well position display 52a and the gel image display 52d displayed on the display unit 52. With this configuration, it is possible to easily recognize what type of abnormality is detected in which object-to-be-measured, in both the well position display 52a and the gel image display 52d. Therefore, it is possible to easily compare the positions of the wells 70a and 71a in which the object-to-be-measured in which an abnormality is detected is placed and the analysis result in the gel image display 52d.

Further, in the present embodiment, as described above, the analysis apparatus 102 is configured to display an abnormality detection indication in the gel image display 52d including the well number 93a indicating the wells 70a and 71a in which the measured objects-to-be-measured are placed, and a measurement order number 93b indicating the measurement order. With this configuration, the gel image display 52d displays the well number 93a and the measurement order number 93b together with the abnormality detection indication, in a case where an abnormality is detected in the measurement and analysis of any of the plurality of objects-to-be-measured, it is possible to easily recognize the position of the well 70a or 71a in which the object-to-be-measured for which the measurement abnormality is detected is placed, and the order of measurement, by viewing the gel image display 52d.

Further, in the present embodiment, as described above, the analysis apparatus 102 is configured to display, on the display unit 52, the gel image display 52d in which the respective analysis results of the plurality of objects-to-be-measured are displayed side by side, display an abnormality detection indication in each of the plurality of analysis results which are displayed side by side in the gel image display 52d, and is able to change the arrangement order of the plurality of analysis results in the gel image display 52d. With this configuration, the arrangement order of the plurality of analysis results displayed side by side in the gel image display 52d can be changed, so that the analysis results can be rearranged for each type of detected abnormality. Therefore, it is possible to easily compare a plurality of analysis results for each type of detected abnormality. Further, since the arrangement order of the plurality of analysis results can be changed in the gel image display 52d, only the analysis results in which no abnormality has been detected can be collectively displayed side by side. Therefore, it is possible to easily compare normal analysis results in which no abnormality is detected.

Further, in the present embodiment, as described above, the electrophoresis system 100 includes an operation unit 51 that receives an input operation, and the analysis apparatus 102 is configured to display on the display unit 52, a well position display 52a indicating the respective positions of the plurality of wells 70a and 71a in a grid pattern so as to correspond to the plurality of wells 70a and 71a arranged in a grid pattern, and display an abnormality detection indication when an abnormality is detected, for each of the plurality of wells 70a and 71a arranged in a grid pattern in the well position display 52a, and is configured to select the analysis result of each of the plurality of objects-to-be-measured placed in the plurality of wells 70a and 71a included in a predetermined area 95, based on the reception of a selection operation by the operation unit 51 to surround the predetermined area 95 from among the plurality of wells 70a and 71a arranged in a grid pattern in the well position display 52a. With this configuration, a plurality of wells 70a and 71a can be easily selected at the same time, by performing a selection operation so as to surround a predetermined area 95 in the well position display 52a by using the operation unit 51. Therefore, the plurality of wells 70a and 71a can be selected more easily than a case where the plurality of wells 70a and 71a are selected one by one.

Effects of Electrophoresis Analysis Method and Electrophoresis Analysis Program According to Present Embodiment

The electrophoresis analysis method and the electrophoresis analysis program 53a of the present embodiment can obtain the following effects.

In the electrophoresis analysis method and the electrophoresis analysis program 53a of the present embodiment, with the configuration described above, in a case where at least one of an apparatus error that is an abnormality in the electrophoresis apparatus 101 and an analysis error that is an abnormality in the analysis of the component of the object-to-be-measured is detected, an abnormality detection indication that allows identification of the type of the detected abnormality is displayed on the display unit 52. Thus, in a case where an abnormality including at least one of an apparatus error and an analysis error is detected, the type of the detected abnormality can be easily identified by viewing the abnormality detection indication displayed on the display unit 52. As a result, it is possible to provide an electrophoresis analysis method and an electrophoresis analysis program 53a capable of easily identifying the type of abnormality, in a case where an abnormality occurs in the analysis result of analyzing the object-to-be-measured separated by electrophoresis.

### Modification Example

In addition, the embodiments disclosed here should be considered to be exemplary and not restrictive in all respects. The scope of the present invention is shown not by the description of the above embodiment but by the scope of claims, and further includes all changes (modification examples) within the meaning and scope equivalent to the scope of claims.

For example, in the above embodiment, an example is shown in which the analysis apparatus 102 acquires the measurement values 111 in real time as the electrophoresis apparatus 101 performs the measurement of the object-to-be-measured, and sequentially analyzes the acquired measurement values 111, but the present invention is not limited to this. In the present invention, the measurement values 111 acquired by the measurement unit 30 of the electrophoresis apparatus 101 may be stored in the storage unit 53 or the like, and analysis may be performed based on the stored measurement values 111. That is, the analysis by the analysis apparatus 102 may be performed at a timing different from the measurement of the object-to-be-measured by the electrophoresis apparatus 101. In that case, the measurement value 111 acquired for each measurement of the object-to-be-measured and the detected abnormality (apparatus error or analysis error) are associated and stored in the storage unit 53 or the like.

Further, in the above-described embodiment, an example is shown in which the analysis apparatus 102 that analyzes the component of the object-to-be-measured is provided separately from the electrophoresis apparatus 101, but the present invention is not limited to this. In the present invention, the electrophoresis apparatus 101 and the analysis apparatus 102 may be integrally formed. Similarly, only the display unit 52 may be formed integrally with the electrophoresis apparatus 101. Further, the display unit 52 may be placed separately while being spaced apart from both the electrophoresis apparatus 101 and the analysis apparatus 102.

Further, in the above-described embodiment, an example is shown in which the analysis error indication 92a indicating that an analysis error has been detected in the abnormality detection indication is shown by an icon image indication, and the serious error indication 92b and the warning error indication 92c indicating that an apparatus error has been detected is shown by color-coding according to a degree of importance, but the present invention is not limited to this. For example, all of the analysis error indication 92a, the serious error indication 92b, and the warning error indication 92c may be displayed so as to be mutually identifiable by an icon image indication, or may be mutually identifiable by color-coded display. Further, the serious error indication 92b and the warning error indication 92c may be made common. Further, the abnormality detection indication may be character information that allows identification of the type of abnormality.

Further, in the above-described embodiment, an example is shown in which an abnormality detection indication is displayed on both the well position display 52a and the gel image display 52d, in a case where at least one of an apparatus error and an analysis error is detected, but the present invention is not limited to this. For example, the abnormality detection indication may be displayed on only one of the well position display 52a and the gel image display 52d.

Further, in the above embodiment, an example of displaying the standby indication 94 in the gel image display 52d is shown, but the present invention is not limited to this. For example, instead of displaying the standby indication 94 on the gel image display 52d, an indication indicating that the measurement is on standby may be displayed only in the well position display 52a.

Further, in the above embodiment, an example is shown in which a serious error which is an apparatus error having a relatively high degree of importance is detected, in a case where the magnitude of the voltage value, the current value, or the internal temperature detected by the abnormality detection unit 80 is greater than the predetermined abnormality determination threshold value, and a warning error which is an apparatus error having a relatively low degree of importance is detected, in a case where the magnitude of the voltage value, the current value, or the internal temperature detected by the abnormality detection unit 80 is not constant (unstable), but the present invention is not limited to this. For example, a serious error may be detected in a case where a communication error, liquid leakage detection, various types of liquid shortage, or the like is detected.

Further, in the above-described embodiment, an example is shown in which the chips 60a to 60c are provided with the preparation channel 63 for guiding the object-to-be-measured to the separation channel 62, but the present invention is not limited to this. For example, the chips 60a to 60c may be configured to have only the separation channel 62 without the preparation channel 63. Further, instead of a shape (cruciform) in which the separation channel 62 and the preparation channel 63 intersect so as to penetrate each other, the preparation channel 63 may intersect the separation channel 62 so as to form a T shape.

Further, in the above-described embodiment, an example is shown in which the electrophoresis apparatus 101 is configured to measure each of the plurality (three) of chips 60a to 60c, but the present invention is not limited to this. For example, one or two chips may be used to measure the object-to-be-measured, or four or more chips may be used. Further, even in a case where the electrophoresis apparatus 101 may be configured to measure each of the three chips 60a to 60c, only one or two chips may be designated (selected) for measurement.

Further, in the above-described embodiment, an example is shown in which the electrophoresis apparatus 101 is configured to perform microchip electrophoresis, but the present invention is not limited to this. For example, it may be configured to perform capillary electrophoresis without using a microchip.

Further, in the above-described embodiment, an example is shown in which the measurement value 111 of the object-to-be-measured is acquired by fluorescence detection, but the present invention is not limited to this. For example, the separated components of the object-to-be-measured may be detected by staining with a reagent.

Aspect

It will be appreciated by those skilled in the art that the exemplary embodiments described above are specific examples of the following aspects.

Item 1

An electrophoresis system including:

an electrophoresis apparatus including a measurement unit that measures an object-to-be-measured separated by electrophoresis in a channel including a separation channel for separating the object-to-be-measured;

an analysis apparatus that analyzes a component of the object-to-be-measured separated by electrophoresis, based on a measurement value of the object-to-be-measured that is measured by the measurement unit; and

a display unit that displays an analysis result of the object-to-be-measured by the analysis apparatus, in which

in a case where at least one of an apparatus error that is an abnormality in the electrophoresis apparatus or an analysis error that is an abnormality in the analysis of the component of the object-to-be-measured is detected, the analysis apparatus is configured to display an abnormality detection indication that allows identification of a type of the detected abnormality, on the display unit.

Item 2

The electrophoresis system according to Item 1, in which

the electrophoresis apparatus is configured to output an apparatus error signal indicating that the apparatus error has been detected, to the analysis apparatus, and

the analysis apparatus is configured to display the abnormality detection indication on the display unit, in at least one of a case where the apparatus error signal is acquired from the electrophoresis apparatus or a case where the analysis error is detected in the analysis of the component of the object-to-be-measured.

Item 3

The electrophoresis system according to Item 1 or 2, in which

in a case where at least one of the apparatus error or the analysis error is detected, the analysis apparatus is configured to display the abnormality detection indication with a different display mode depending on the type of detected abnormality on the display unit.

Item 4

The electrophoresis system according to any one of Items 1 to 3, in which

in a case where the apparatus error is detected, the analysis apparatus is configured to display, on the display unit, the abnormality detection indication with a different display mode depending on a degree of importance of the detected apparatus error.

Item 5

The electrophoresis system according to Item 3 or 4, in which

in a case where at least one of the apparatus error or the analysis error is detected, the analysis apparatus is configured to display, on the display unit, the abnormality detection indication that allows identification of the type of abnormality, by at least one of color-coding according to the type of the detected abnormality or an icon image indication.

Item 6

The electrophoresis system according to any one of Items 1 to 5, in which

the electrophoresis apparatus is configured to measure a plurality of the objects-to-be-measured, and

in a case where at least one of the apparatus error or the analysis error is detected in any one of the plurality of objects-to-be-measured, the analysis apparatus is configured to display the abnormality detection indication so as to allow identification of the object-to-be-measured in which the abnormality is detected.

Item 7

The electrophoresis system according to Item 6, in which

the electrophoresis apparatus is configured to sequentially measure the plurality of objects-to-be-measured, and

the analysis apparatus is configured to display, on the display unit, a standby indication indicating the object-to-be-measured that is waiting for measurement among the plurality of objects-to-be-measured.

Item 8

The electrophoresis system according to Item 6 or 7, in which

the analysis apparatus is configured to

display, on the display unit, a well position display indicating a position of each of a plurality of wells in which each of the plurality of objects-to-be-measured is placed, and a gel image display indicating the analysis result of each of the plurality of objects-to-be-measured, and

display the abnormality detection indication that allows identification of the object-to-be-measured in which the abnormality is detected, in the well position display and the gel image display displayed on the display unit.

Item 9

The electrophoresis system according to Item 8, in which

the analysis apparatus is configured to display the abnormality detection indication in the gel image display including a well number indicating the well in which the measured object-to-be-measured is placed and a measurement order number indicating the order of measurement.

Item 10

The electrophoresis system according to Item 8 or 9, in which

the analysis apparatus is configured to

display the gel image display in which the respective analysis results of the plurality of objects-to-be-measured are displayed side by side, on the display unit, and

display the abnormality detection indication, and make an arrangement order of the plurality of analysis results in the gel image display changeable, in each of the plurality of analysis results displayed side by side in the gel image display.

Item 11

The electrophoresis system according to any one of Items 8 to 10, further including:

an operation unit that receives an input operation, in which

the analysis apparatus is configured to

display, on the display unit, the well position display indicating respective positions of the plurality of wells in a grid pattern so as to correspond to the plurality of wells arranged in a grid pattern, and

display the abnormality detection indication when an abnormality is detected, for each of the plurality of wells arranged in a grid pattern in the well position display, and

is configured to select the analysis result of each of the plurality of objects-to-be-measured placed in the plurality of wells included in a predetermined area, based on the reception of a selection operation by the operation unit to surround the predetermined area from among the plurality of wells arranged in a grid pattern in the well position display.

Item 12

An electrophoresis apparatus including:

a measurement unit that measures an object-to-be-measured separated by electrophoresis in a channel including a separation channel for separating the object-to-be-measured, in which

in a case where at least one of an apparatus error that is an abnormality in the electrophoresis apparatus or an analysis error that is an abnormality in analysis of a component of the object-to-be-measured separated by electrophoresis based on a measurement value of the object-to-be-measured that is measured by the measurement unit is detected, the electrophoresis apparatus is configured to display an abnormality detection indication that allows identification of a type of the detected abnormality, on a display unit.

Item 13

An electrophoresis analysis method including:

a step of analyzing a component of an object-to-be-measured separated by electrophoresis, based on a measurement value obtained by measuring the object-to-be-measured separated by electrophoresis in a channel including a separation channel for separating the object-to-be-measured; and

a step of, in a case where at least one of an apparatus error that is an abnormality in the electrophoresis apparatus that measures the object-to-be-measured separated by electrophoresis or an analysis error that is an abnormality in analysis of the component of the object-to-be-measured is detected, displaying an abnormality detection indication that allows identification of a type of the detected abnormality, on a display unit.

Item 14

An electrophoresis analysis program causing a computer to execute:

a step of analyzing a component of an object-to-be-measured separated by electrophoresis, based on a measurement value obtained by measuring the object-to-be-measured separated by electrophoresis in a channel including a separation channel for separating the object-to-be-measured; and

a step of, in a case where at least one of an apparatus error that is an abnormality in the electrophoresis apparatus that measures the object-to-be-measured separated by electrophoresis or an analysis error that is an abnormality in analysis of the component of the object-to-be-measured is detected, displaying an abnormality detection indication that allows identification of a type of the detected abnormality, on a display unit.

What is claimed is:

1. An electrophoresis system comprising:
an electrophoresis apparatus including a measurement unit that measures an object-to-be-measured separated by electrophoresis in a channel including a separation channel for separating the object-to-be-measured;
an analysis apparatus that analyzes a component of the object-to-be-measured separated by electrophoresis, based on a measurement value of the object-to-be-measured that is measured by the measurement unit; and
a display unit that displays an analysis result of the object-to-be-measured by the analysis apparatus, wherein
in a case where at least one of an apparatus error that is an abnormality in the electrophoresis apparatus or an analysis error that is an abnormality in the analysis of the component of the object-to-be-measured is detected, the analysis apparatus is configured to display an abnormality detection indication that allows identification of a type of the detected abnormality, on the display unit.

2. The electrophoresis system according to claim 1, wherein
the electrophoresis apparatus is configured to output an apparatus error signal indicating that the apparatus error is detected to the analysis apparatus, and
the analysis apparatus is configured to display the abnormality detection indication on the display unit, in at least one of a case where the apparatus error signal is acquired from the electrophoresis apparatus or a case where the analysis error is detected in the analysis of the component of the object-to-be-measured.

3. The electrophoresis system according to claim 1, wherein
in a case where at least one of the apparatus error or the analysis error is detected, the analysis apparatus is configured to display the abnormality detection indication with a different display mode depending on the type of detected abnormality on the display unit.

4. The electrophoresis system according to claim 1, wherein
in a case where the apparatus error is detected, the analysis apparatus is configured to display, on the display unit, the abnormality detection indication with a different display mode depending on a degree of importance of the detected apparatus error.

5. The electrophoresis system according to claim 3, wherein
in a case where at least one of the apparatus error or the analysis error is detected, the analysis apparatus is configured to display, on the display unit, the abnormality detection indication that allows identification of the type of abnormality, by at least one of color-coding or an icon image indication according to the type of the detected abnormality.

6. The electrophoresis system according to claim 1, wherein
the electrophoresis apparatus is configured to measure a plurality of the objects-to-be-measured, and
in a case where at least one of the apparatus error or the analysis error is detected in any one of the plurality of objects-to-be-measured, the analysis apparatus is configured to display the abnormality detection indication so as to allow identification of the object-to-be-measured in which the abnormality is detected.

7. The electrophoresis system according to claim 6, wherein
the electrophoresis apparatus is configured to sequentially measure the plurality of objects-to-be-measured, and
the analysis apparatus is configured to display, on the display unit, a standby indication indicating the object-to-be-measured that is waiting for measurement among the plurality of objects-to-be-measured.

8. The electrophoresis system according to claim 6, wherein
the analysis apparatus is configured to
display, on the display unit, a well position display indicating a position of each of a plurality of wells in which each of the plurality of objects-to-be-measured is placed, and a gel image display indicating the analysis result of each of the plurality of objects-to-be-measured, and
display the abnormality detection indication that allows identification of the object-to-be-measured in which the abnormality is detected, in the well position display and the gel image display displayed on the display unit.

9. The electrophoresis system according to claim 8, wherein
the analysis apparatus is configured to display the abnormality detection indication in the gel image display including a well number indicating the well in which the measured object-to-be-measured is placed and a measurement order number indicating the order of measurement.

10. The electrophoresis system according to claim 8, wherein
the analysis apparatus is configured to
display the gel image display in which the respective analysis results of the plurality of objects-to-be-measured are displayed side by side, on the display unit, and
display the abnormality detection indication in each of the plurality of analysis results displayed side by side in the gel image display, and make an arrangement order of the plurality of analysis results in the gel image display changeable.

11. The electrophoresis system according to claim 8, further comprising:
an operation unit that receives an input operation, wherein
the analysis apparatus is configured to display, on the display unit, the well position display indicating respective positions of the plurality of wells in a grid pattern so as to correspond to the plurality of wells arranged in a grid pattern, and display the abnormality detection indication when an abnormality is detected, for each of the plurality of wells arranged in a grid pattern in the well position display, and
is configured to select the analysis result of each of the plurality of objects-to-be-measured placed in the plurality of wells included in a predetermined area, based on the reception of a selection operation by the operation unit to surround the predetermined area from among the plurality of wells arranged in a grid pattern in the well position display.

12. An electrophoresis apparatus comprising:
a measurement unit that measures an object-to-be-measured separated by electrophoresis in a channel including a separation channel for separating the object-to-be-measured, wherein
in a case where at least one of an apparatus error that is an abnormality in the electrophoresis apparatus or an analysis error that is an abnormality in analysis of a component of the object-to-be-measured separated by electrophoresis based on a measurement value of the object-to-be-measured that is measured by the measurement unit is detected, the electrophoresis apparatus is configured to display an abnormality detection indication that allows identification of a type of the detected abnormality, on a display unit.

13. An electrophoresis analysis method comprising:
a step of analyzing a component of an object-to-be-measured separated by electrophoresis, based on a measurement value obtained by measuring the object-to-be-measured separated by electrophoresis in a channel including a separation channel for separating the object-to-be-measured; and
a step of, in a case where at least one of an apparatus error that is an abnormality in an electrophoresis apparatus that measures the object-to-be-measured separated by electrophoresis or an analysis error that is an abnormality in analysis of the component of the object-to-be-measured is detected, displaying an abnormality detection indication that allows identification of a type of the detected abnormality, on a display unit.
